(19) United States
(12) Patent Application Publication
(10) Pub. No.: US 2008/0213176 A1
(43) Pub. Date: Sep. 4, 2008

(54) Compositions for Targeting Amine Oxidases in Vivo

(76) Inventors: Sirpa Jalkanen, Piispanristi (FI); Marko Salmi, Turku (FI)

Correspondence Address: ROTHWELL, FIGG, ERNST & MANBECK, P.C., 1425 K STREET, N.W., SUITE 800, WASHINGTON, DC, 20005, US

(21) Appl. No.: 11/915,410

(22) PCT Filed: May 4, 2006
(86) PCT No.: PCT/FI2006/000144

§ 371 Date: Nov. 26, 2007

(30) Foreign Application Priority Data
May 31, 2005 (FI) 20050574

(51) Int. Cl. A61K 51/00; A61K 49/00; C07K 7/00; A61P 43/00

(52) U.S. Cl. 424/1.69; 424/9.1; 530/328

(57) ABSTRACT

This invention relates to a diagnostic compositions for targeting an amine oxidase enzyme in vivo, said composition comprising a labelled peptide having the capability to bind to said enzyme. Further, the invention concerns a pharmaceutical composition for use to modulate the activity of an amine oxidase enzyme, said composition comprising a peptide having the capability to bind to said enzyme, wherein said peptide further is conjugated to a therapeutically active agent. Still further, the invention concerns a novel peptide and its uses, especially as VAP-1 inhibitor.

### FIELD OF THE INVENTION

This invention relates to diagnostic and pharmaceutical compositions for targeting amine oxidases, particularly vascular adhesion protein-1 (VAP-1) in vivo, and to the use thereof for diagnosing or treating or preventing diseases or conditions related to said amine oxidases. Moreover, the invention concerns a novel lysine containing peptide and its use for modulating the activity of amine oxidases and/or for targeting amine oxidases.

### BACKGROUND OF THE INVENTION

The publications and other materials used herein to illuminate the background of the invention, and in particular, cases to provide additional details respecting the practice, are incorporated by reference.

VAP-1 is a human endothelial cell adhesion molecule that has several unique properties that distinguish it from the other inflammation-related adhesion molecules. It has a unique and restricted expression pattern and mediates lymphocyte binding to vascular endothelium (Salmi, M., and Jalkanen, S., Science 257:1407-1409 (1992)). Inflammation induces the upregulation of VAP-1 to the surface of vascular endothelial cells mediating leukocyte entry to skin, gut and inflamed synovium (Salmi, M., and Jalkanen, S., Science 257:1407-1409 (1992); Salmi, M, et al., J. Exp. Med. 178:2255-2260 (1993); Arvillommi, A., et al., Eur. J. Immunol. 26:825-833 (1996); Salmi, M., et al., J. Clin. Invest. 99:2165-2172 (1997): (Salmi. M., and Jalkanen, S., J. Exp. Med. 183:569-579 (1996); J. Exp. Med. 186:589-600 (1997)). One of the most interesting features of VAP-1 is a catalytic extracellular domain which contains a monoamine oxidase activity (Smith, D. J., et al., J. Exp. Med. 188:17-27 (1998)).

The cloning and sequencing of the human VAP-1 cDNA revealed that it encodes a transmembrane protein with homology to a class of enzymes called the copper-containing amine oxidases (E.C. 1.4.3.6). Enzyme assays have shown that VAP-1 possesses a monoamine oxidase (MAO) activity which is present in the extracellular domain of the protein (Smith, D. J., et al., J. Exp. Med. 188:17-27 (1998)). Thus, VAP-1 is an ecto-enzyme. Analysis of the VAP-1 MAO activity showed that VAP-1 belongs to the class of membrane-bound MAO's termed semicarbazide-sensitive amine oxidases (SSAO). These are distinguished from the widely distributed mitochondrial MAO-A and B flavoproteins by amino acid sequence, cofactor, substrate specificity and sensitivity to certain inhibitors. However, certain substrates and inhibitors are common to both SSAO and MAO activities. The mammalian SSAO's can metabolize various monoamines produced endogenously or absorbed as dietary or xenobiotic substances. They act principally on primary aliphatic or aromatic monoamines such as methylamine or benzylamine (Lyles G. A., Int. J. Biochem. Cell Biol, 28:259-274 (1996)). Thus, VAP-1 located on the vascular endothelial cell surface can act on circulating primary monoamines with the following reaction pathway.

RNH₂+O₂+H₂O------->RCHO+H₂O₂+NH₃

The physiological substrates of VAP-1 SSAO in man have not been clearly identified. However, methylamine is a good substrate for VAP-1 SSAO. Methylamine is a product of various human biochemical pathways for the degradation of creatinine, sarcosine and adrenaline, and is found in various mammalian tissues and in blood. It can also be derived from the diet by gut bacterial degradation of dietary precursors. The concentration of methylamine in the blood can be increased in certain physiological and pathological situations such as diabetes. Another potential physiological substrate is aminoacetone.

VAP-1 SSAO activity has been proposed to be directly involved in the pathway of leukocyte adhesion to endothelial cells by a novel mechanism involving direct interaction with an amine substrate presented on a VAP-1 ligand expressed on the surface of a leukocyte (Salmi et al. Immunity, (2001)). This publication describes the direct involvement of VAP-1 SSAO activity in the process of adhesion of leukocytes to endothelium. Thus inhibitors of VAP-1 SSAO activity could be expected to reduce leukocyte adhesion in areas of inflammation and thereby reduce leukocyte trafficking into the inflamed region and therefore the inflammatory process itself.

In human clinical tissue samples expression of VAP-1 is induced at sites of inflammation. This increased level of VAP-1 can lead to increased production of H₂O₂ generated from the action of the VAP-1 SSAO extracellular domain on monoamines present in the blood. This generation of H₂O₂ in the localized environment of the endothelial cell could initiate other cellular events. H₂O₂ is a known signaling molecule that can upregulate other adhesion molecules and this increased adhesion molecule expression may lead to enhanced leukocyte trafficking into areas in which VAP-1 is expressed. It also may be that other products of the VAP-1 SSAO reaction could have biological effects also contributing to the inflammatory process. Thus the products of the VAP-1 SSAO activity may be involved in an escalation of the inflammatory process which could be blocked by specific SSAO inhibitors.

VAP-1 SSAO may be involved in a number of other pathological conditions associated with an increased level of circulating amine substrates of VAP-1 SSAO. The oxidative deamination of these substrates would lead to an increase in the level of toxic aldehydes and oxygen radicals in the local environment of the endothelial cell which could damage the cells leading to vascular damage. Increased levels of methylamine and aminoacetone have been reported in patients with Type I and Type II diabetes and it has been proposed that the vasculopathies such as retinopathy, neuropathy and nephropathy seen in late stage diabetes could be treated with specific inhibitors of SSAO activity.

Grifantini, M., et al., Farmaco, Ed. Sci. 23(3):197-203 (1968), report the synthesis of several alkyl- and acyl-derivatives of N-amino-1-ephedrine and N-amino-d-pseudoephedrine having antidepressant and monoamine oxidase inhibitory properties.

Jeffrey O'Sullivan et al., Biochimica et Biophysica Acta 1647 (2003) 367-371 report the inhibition of semicarbazide-sensitive amine oxidases by certain aminohexoses, namely glucosamine, galactosamine and mannosamine.

The international patent publications WO 02/020290 and WO 03/006003 disclose certain hydrazino compounds useful as specific VAP-1 SSAO inhibitors that modulate VAP-1 activity. These compounds are described as useful for the treatment of acute and chronic inflammatory conditions or diseases as well as diseases related to carbohydrate metabolism, aberrations in adipocyte differentiation or function and smooth muscle cell function, and various vascular diseases.

The publication Yegutin G G et al. Eur J. Immunol. 2004 August; 34(8), 2276-85 discloses a group of lysine containing peptides of 7 to 9 amino acids that fit within the groove on the surface of VAP-1 and can present free NH₂ groups via the narrow substrate channel into the catalytically active site of VAP-1. It was shown that such polypeptides inhibit the enzymatic activity of VAP-1 and, importantly, diminish lymphocyte-endothelial interactions under physiologically relevant flow conditions. Computer docking simulations showed that certain peptides fit well to the active site channel of VAP-1. Peptides GGGGKGGGG (SEQ ID NO 1), GGGKGGG (SEQ ID NO 2), GGGKEGG (SEQ ID NO 3) and GGGKKGG (SEQ ID NO 4) which all contain a lysine residue in the middle of a flexible polypeptide were found to bind optimally to the active site of VAP-1. The side chain of lysine is long enough for the amino group to interact with the topa-quinone residue of VAP-1 (Tyr471 of VAP-1 is autocatalytically modified to a topa-quinone) during the catalytic reaction. The flexible peptide backbone in a V-shaped conformation of these peptides is able to interact extensively with the active site cavity filling the groove on the VAP-1 surface. In contrast, a bulky and rigid peptide without a lysine residue, peptides containing a lysine residue in the middle position of a bulkier alanine-based peptide or at terminal positions in a flexible glycine-based peptide as well as peptides containing a middle arginine instead of lysine could neither fit properly within the substrate channel or interact efficiently with the VAP-1 structure.

However, the aforementioned publication does not disclose or suggest use of the peptides in diagnostic compositions or in pharmaceutical compositions as carriers for therapeutically active agents.

### OBJECTS AND SUMMARY OF THE INVENTION

One object of the present invention is to provide a diagnostic composition useful for in vivo location of VAP-1 or other amine oxidases. The knowledge of the precise location of an amine oxidase will be useful to specifically direct the various treatment methods and other measures to the tissues in which the amine oxidase occur.

Another object is to provide a pharmaceutical composition having a carrier specifically targeting said amine oxidase. A therapeutically active agent conjugated to the carrier is useful for exact local treatment of tissues influenced by the amine oxidase and the pharmaceutical composition is thus aimed for exact local treatment or prevention of amine oxidase related diseases or disorders.

Thus, according to one aspect, this invention concerns a diagnostic composition for targeting an amine oxidase enzyme in vivo, said composition comprising a labelled peptide having the capability to bind to said enzyme, and wherein the peptide comprises an amino acid sequence of at least 7 amino acids, said sequence comprising a mid-part having at least one lysine residue in said mid-part of the sequence, and terminal parts, each terminal part preferably comprising at least two consecutive glycine residues.

According to another aspect, the invention concerns a method for diagnosing amine oxidase related diseases or conditions in a mammal in vivo, said method comprising administering to the mammal a composition according to this invention, and detecting the label.

According to a third aspect, the invention concerns a pharmaceutical composition for use to modulate the activity of an amine oxidase enzyme, said composition comprising a peptide having the capability to bind to said enzyme, wherein said peptide further is conjugated to a therapeutically active agent, and wherein the peptide comprises an amino acid sequence of at least 7 amino acids, said sequence comprising a mid-part having at least one lysine residue in said mid-part of the sequence, and terminal parts, each terminal part preferably comprising at least two consecutive glycine residues.

According to a fourth aspect, the invention concerns a method for the treatment of or prevention of an amine oxidase related disease or condition in a mammal, said method comprising administering to the mammal a pharmaceutical composition according to this invention.

According to a fifth aspect, the invention concerns a novel peptide, its use to modulate the activity of an amine oxidase enzyme in vivo, or in diagnostic or pharmaceutical compositions for targeting an amine oxidase in vivo.

### DETAILED DESCRIPTION OF THE INVENTION

### Definitions

The term “treatment” or “treating” shall be understood to include complete curing of a disease or condition, as well as amelioration or alleviation of said disease or condition.

The term “prevention” shall be understood to include complete prevention, prophylaxis, as well as lowering the individual's risk of falling ill with said disease or condition.

The term “mammal” refers to a human or animal subject.

The term “therapeutically active agent agent” shall here be understood to cover any geometric isomer, stereoisomer, diastereoisomer, racemate or any mixture of isomers, and any pharmaceutically acceptable salt of the compound.

### PREFERABLE EMBODIMENTS

### Preferable Peptides:

Preferably, the peptide comprises an amino acid sequence of at least 7 amino acids. The sequence comprises a mid-part with at least one lysine residue in said mid-part of the sequence, and terminal parts, each terminal part preferably comprising at least two consecutive glycine residues.

The peptide can in principle be much longer than 7 amino acids. However, very long chains increase the possibility of configuration changes, such as folding, and therefore we believe that a peptide with an amino acid sequence of 7 to 9 amino acids would be most preferable.

Particularly useful sequences are those selected from the group consisting of

| | |
|---|---|
| GGGGKGGGG, | (SEQ ID NO 1) |
| GGGKGGG, | (SEQ ID NO 2) |
| GGGKEGG, | (SEQ ID NO 3) |
| GGGKKGG | (SEQ ID NO 4) |
| and | |
| GGGGKYGGG. | (SEQ ID NO 5) |

Most preferably, the peptide is GGGGKYGGG (SEQ ID NO 5), which is a novel peptide. The intrinsic VAP-1 inhibiting activity of this peptide has been found to be stronger than that of the previously disclosed lysine containing peptides.

### Preferable Labels:

In diagnostic compositions, the label can be any detectable label suitable for use in vivo. Thus, the label could be, for example, a fluorescent label or more preferably, a radioisotope.

We believe that the peptides disclosed in this invention are capable of binding to amine oxidases generally. However, they are especially useful for binding to VAP-1. Therefore, the diagnostic composition is particularly useful for diagnosing VAP-1 related disease or disorders; most preferably for detecting locations of inflammations related to VAP-1.

### Preferable Pharmaceutical Compositions:

The peptides as such are intrinsically more or less capable of modulating amine oxidase activities as inhibitors or substrates. The therapeutically active agent conjugated to the peptide can be any drug. Most preferably, the therapeutically active agent is an amine oxidase inhibitor or substrate, i.e. a VAP-1 inhibitor or VAP-1 substrate. If, for example, the peptide as such is a VAP-1 inhibitor, the conjugated therapeutically active agent is also a VAP-1 inhibitor.

The drug molecule can be conjugated to the peptide in manners known as such by using a suitable coupling group. Such groups can e.g. be amino, imino, amido, imido, thio, carbonyl, carboxyl etc. groups and derivatives of said groups. As example of one suitable specific conjugating group can be mentioned 1-ethyl-3-(3-dimethyl-aminopropyl)carbidiimide or N-hydroxysuccinimide, which have been used to couple doxorubicin to peptides (W Arap et al., Science Vol. 279, 16 Jan. 1998, pp. 377-380). If needed, a linker group could also be inserted e.g. between the peptide and the aforementioned coupling group in order to facilitate coupling that otherwise would be difficult due to steric hindrance or other reasons. Such linkers are well known in the art. In its simplest form, the linker can be a hydrocarbon chain of suitable length.

Diseases or Conditions with Responsiveness to Amine Oxidase Inhibitors:

As examples of groups of diseases or conditions the treatment or prevention of which would benefit from inhibiting amine oxidase enzyme can be mentioned inflammatory diseases or conditions; diseases related to carbohydrate metabolism; diseases related to aberrations in adipocyte differentiation or function or smooth muscle cell function and vascular diseases. However, the diseases or conditions are not restricted to these groups.

According to one embodiment, the inflammatory disease or condition can be a connective tissue inflammatory disease or condition, such as, but not limited to ankylosing spondylitis, Reiter's syndrome, psoriatic arthritis, osteoarthritis or degenerative joint disease, rheumatoid arthritis, Sjögren's syndrome, Bechet's syndrome, relapsing polychondritis, systemic lupus erythematosus, discoid lupus erythematosus, systemic sclerosis, eosinophilic fasciitis, polymyositis and dermatomyositis, polymyalgia rheumatica, vasculitis, temporal arteritis, polyarterisis nodosa, Wegner's granulamatosis, mixed connective tissue disease, or juvenile rheumatoid arthritis.

According to another embodiment, said inflammatory disease or condition is a gastrointestinal inflammatory disease or condition, such as, but not limited to Crohn's disease, ulcerative colitis, irritable bowel syndrome (spastic colon), fibrotic conditions of the liver, inflammation of the oral mucosa (stomatitis), or recurrent aphtous stomatitis.

According to a third embodiment, said inflammatory disease or condition is a central nervous system inflammatory disease or condition, such as, but not limited to multiple sclerosis, Alzheimer's disease, or ischemia-reperfusion injury associated with ischemic stroke.

According to a fourth embodiment, said inflammatory disease or condition is a pulmonary inflammatory disease or condition, such as, but not limited to asthma, chronic obstructive pulmonary disease, or adult respiratory distress syndrome.

According to a fifth embodiment, said inflammatory disease or condition is a skin inflammatory disease or condition such as, but not limited to contact dermatitis, atopic dermatitis, psoriasis, pityriasis rosea, lichen planus, or pityriasis rubra pilaris.

According to a seventh embodiment said inflammatory condition is related to tissue trauma or resulting from organ transplantations or other surgical operations.

According to an eighth embodiment, said disease related to carbohydrate metabolism is a disease such as but not limited to diabetes, atherosclerosis, vascular retinopathies, retinopathy, nephropathy, nephrotic syndrome, polyneuropathy, mononeuropathies, autonomic neuropathy, foot ulcers or joint problems.

According to a tenth embodiment said disease relating to aberrations in adipocyte differentiation or function or smooth muscle cell function is a disease such as but not limited to atherosclerosis or obesity.

According to an eleventh embodiment, the vascular disease is a disease such as but not limited to atheromatous ateriosclerosis, nonatheromateous ateriosclerosis, ischemic heart disease, peripheral aterial occlusion, thromboangiitis obliterans (Buerger's disease), or Raynaud's disease and phenomenon.

For the purpose of this invention, the compounds disclosed in this invention or their isomer, isomer mixture or their pharmaceutically acceptable salts can be administered by various routes. For example, administration can be by parenteral, subcutaneous, intravenous, intraarticular, intrathecal, intramuscular, intraperitoneal, or intradermal injections, or by transdermal, buccal, oromucosal, ocular routes or via inhalation. Alternatively, or concurrently, administration can be by the oral route. Particularly preferred is oral administration. Suitable oral formulations include e.g. conventional or slow-release tablets and gelatine capsules.

The required dosage of the compounds will vary with the particular disease or condition being treated, the severity of the condition, the duration of the treatment, the administration route and the specific compound being employed.

Thus, a typical dose is in the dosage range of about 0.1 microgram/kg to about 300 mg/kg, preferably between 1.0 microgram/kg to 10 mg/kg body weight. Compounds of the present invention may be administered in a single daily dose, or the total daily dosage may be administered in divided doses of two, three or four times daily.

The invention will be illuminated by the following non-restrictive Experimental Section.

### Experiments

### Materials and Methods

Cells. The CHO cells stably transfected with the cDNA encoding VAP-1 was used. The VAP-1 expression level of these cells was confirmed by staining with anti-VAP-1 monoclonal antibodies and subsequent flow cytometric analyses. The VAP-1 transfectants were grown into the confluence, detached, counted and equal numbers were divided to 96 well plates for enzymatic assays.

Fluorometric detection of SSAO-mediated H₂O₂ formation. Amine oxidase activities were measured using a highly sensitive and stable probe for H₂O₂ Amplex Red reagent (Molecular Probes Europe BV) as described previously (Salmi et al., 2001). The enzymes were incubated in white microplates in the final volume of 200 μl KRPG containing various concentrations of the tested peptides, 0.8 U/ml horseradish peroxidase, 60 μM Amplex Red reagent, and methylamine as preferred amine substrate for SSAO. Fluorescence intensity of the samples was measured using Tecan ULTRA fluoropolarometer (excitation/emission wavelengths 545/595 nm). To evaluate the amount of H₂O₂ formed via SSAO-mediated reaction, specific enzyme inhibitor semicarbazide (200 μM) was included in the control well, and these values were subtracted from the total amount of H₂O₂ formed. To test whether the peptides could interfere with fluorometric H₂O₂ detection, H₂O₂ concentrations were calculated from calibration curves generated by serial dilutions of standard H₂O₂ in the presence of various concentrations of peptides. All calibration curves show linear dependence of the measured fluorescence intensity vs. H₂O₂ concentration in the presence of peptides in concentrations less than 250 micrograms/ml. Thus, the peptides in 10 and 100 micrograms/ml did not interfere with H₂O₂ measurements.

### Results

The inhibitory activity of peptide GGGGKYGGG (SEQ ID NO 5) was tested in concentrations 10 and 100 micrograms/ml and it was compared to the activity of the best peptide known earlier (GGGGKGGGG; SEQ ID NO 1). GGGGKYGGG (SEQ ID NO 5) inhibited 42.8% of the activity at concentration 100 micrograms/ml and 33.5% at 10 micrograms/ml, while the corresponding figures for GGGGKGGGG (SEQ ID NO 1) were 20.3% and 17.4%, respectively. These results indicate that GGGGKYGGG (SEQ ID NO 5) is a more potent inhibitor of VAP-1 than GGGGKGGGG (SEQ ID NO 1) and is presumably also a better tool to for targeting VAP-1 in different applications.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A diagnostic composition for targeting an amine oxidase enzyme in vivo, said composition comprising a labelled peptide having the capability to bind to said enzyme, and wherein the peptide comprises an amino acid sequence of at least 7 amino acids, said sequence comprising a mid-part having at least one lysine residue in said mid-part of the sequence, and terminal parts, each terminal part preferably comprising at least two consecutive glycine residues.

2. The composition according to claim 1 wherein the amine oxidase enzyme is vascular adhesion protein-1 (VAP-1).

3. The composition according to claim 1 wherein said amino acid sequence has 7 to 9 amino acids.

4. The composition according to claim 3 wherein said amino acid sequence is selected from the group consisting of

| | |
|---|---|
| a) GGGGKGGGG | (SEQ ID NO 1) |
| b) GGGKGGG | (SEQ ID NO 2) |
| c) GGGKEGG | (SEQ ID NO 3) |
| d) GGGKKGG | (SEQ ID NO 4) |
| and | |
| e) GGGGKYGGG. | (SEQ ID NO 5) |

5. The composition according to claim 1 wherein the label is a radioisotope.

6. A method for diagnosing amine oxidase related diseases or conditions in a mammal in vivo, said method comprising administering to the mammal a composition according to claim 1, and detecting the label.

7. The method according to claim 6 wherein the amine oxidase is VAP-1.

8. The method according to claim 7 wherein locations of inflammation is detected.

9. A pharmaceutical composition for use to modulate the activity of an amine oxidase enzyme, said composition comprising a peptide having the capability to bind to said enzyme, wherein said peptide further is conjugated to a therapeutically active agent, and wherein the peptide comprises an amino acid sequence of at least 7 amino acids, said sequence comprising a mid-part having at least one lysine residue in said mid-part of the sequence, and terminal parts, each terminal part preferably comprising at least two consecutive glycine residues.

10. The composition according to claim 9 wherein the amine oxidase enzyme is VAP-1.

11. The composition according to claim 10 wherein the therapeutically active agent is a VAP-1 inhibitor or VAP-1 substrate.

12. The composition according to claim 9 wherein said amino acid sequence has 7 to 9 amino acids.

13. The composition according to claim 12 wherein said amino acid sequence is selected from the group consisting of

| | |
|---|---|
| a) GGGGKGGGG | (SEQ ID NO 1) |
| b) GGGKGGG | (SEQ ID NO 2) |
| c) GGGKEGG | (SEQ ID NO 3) |
| d) GGGKKGG | (SEQ ID NO 4) |
| and | |
| e) GGGGKYGGG. | (SEQ ID NO 5) |

14. A method for the treatment of or prevention of an amine oxidase related disease or condition in a mammal, said method comprising administering to the mammal a pharmaceutical composition according to claim 9.

15. The method according to claim 14 wherein the amine oxidase is VAP-1.

16. The method according to claim 15 wherein the disease is an inflammatory disease and the peptide is conjugated to a VAP-1 inhibitor.

17. A novel peptide consisting of the amino acid sequence GGGGKYGGG (SEQ ID NO 5).

18-20. (canceled)


SEQUENCE LISTING (5 sequences)

SEQ ID NO: 1 - protein, 9 aa, Artificial
  VAP-1 targeting peptide
GGGGKGGGG

SEQ ID NO: 2 - protein, 7 aa, Artificial
  VAP-1 targeting peptide
GGGKGGG

SEQ ID NO: 3 - protein, 7 aa, Artificial
  VAP-1 targeting peptide
GGGKEGG

SEQ ID NO: 4 - protein, 7 aa, Artificial
  VAP-1 targeting peptide
GGGKKGG

SEQ ID NO: 5 - protein, 9 aa, Artificial
  VAP-1 targeting peptide
GGGGKYGGG